(19) United States
(12) Patent Application Publication
(10) Pub. No.: US 2014/0314873 A1
(43) Pub. Date: Oct. 23, 2014

(54) PROCESS AND COMPOSITION OF A GEL FOR WOUND DISINFECTION AND PROMOTION OF HEALING

(71) Applicant: STAGE ONE TECHNOLOGY PARTNERSHIP, LLC, San Marcos, TX (US)

(72) Inventors: GARY W. BEAL, SAN MARCOS, TX (US); DAVID COCKE, TEMPLE, TX (US)

(73) Assignee: STAGE ONE TECHNOLOGY PARTNERSHIP, LLC, San Marcos, TX (US)

(21) Appl. No.: 14/180,048

(22) Filed: Feb. 13, 2014

Related U.S. Application Data
(60) Provisional application No. 61/763,980, filed on Feb. 13, 2013.

(51) Int. Cl. A61K 47/02; A61K 9/06; A61K 33/40

(52) U.S. Cl. CPC A61K 47/02; A61K 33/40; A61K 9/06
USPC 424/616

(57) ABSTRACT

A process and composition of a gel that is effective at disinfecting wounds and killing fungal infections such as athletes foot and nail fungus and promoting healing in wounds of the skin has been developed. The invention is produced by a process of gelling hydrogen peroxide with synthetic smectite clay nanoparticles that are free of any heavy metals. Unexpectedly the gel actually promotes healing in wounds of the skin.

### CROSS-REFERENCE TO RELATED APPLICATIONS

This applications claims priority to [[U.S. Provisional Patent Application Ser. No. 61/763980, filed Feb. 13, 2013, which is hereby incorporated by reference in its entirety.

### BACKGROUND OF THE INVENTION

It is well established that hydrogen peroxide has excellent disinfecting powers. There are many examples of compositions being utilized for disinfecting surgical equipment, hard surfaces in homes, toilets, drinking water, and many other applications. It is well understood that the disinfection effect of hydrogen peroxide takes substantial time to kill bacteria and fungi. This requires the peroxide to be in contact with the bacteria or fungi for an extended period. This is normally accomplished by soaking the contaminated area in an aqueous solution of the hydrogen peroxide or by wiping or spraying the surface with the solution and allowing it to evaporate, or by applying cloth soaked in the hydrogen peroxide to hold it in place long enough to disinfect. All of these methods are inconvenient and for wounds simple irrigation does not allow enough time.

One method that has been tried that overcomes many of the problems associate with applying hydrogen peroxide is to form a gel that will stay in place. These gels have been utilized to bleach hair, whiten teeth, disinfect surfaces, and oral rinse. There are a number of synthetic and natural polymers that are utilized that will form a stable gel with hydrogen peroxide. These gelling agents are effective at keeping the hydrogen peroxide in place but suffer from forming a sticky film as the water evaporates that is not pleasant to the touch and may also stain clothing. This coating will also build up on surfaces with repeated use. Smectic clays would be a better choice as a thickener however all natural and most synthetic smectites contain substantial amounts of heavy metal contaminants that catalyze the decomposition of hydrogen peroxide.

In the medical field various hydrogen peroxide containing compositions have been utilized to disinfect implants prior to implantation or as bandages on burns or other injuries to the skin. In the case of direct application of hydrogen peroxide to burns or other injuries to the skin the concentration of peroxide is kept below 0.1%. The reason for the low level of hydrogen peroxide is that the medical literature clearly recommends not putting more than trace amounts of hydrogen peroxide in a wound because it will disinfect the wound but it will also kill good tissue and retard healing.

It is therefore desirable to produce a hydrogen peroxide gel that is stable upon storage, doesn't leave a tacky residue, and that actually disinfects but also promotes healing.

### BRIEF SUMMARY OF THE INVENTION

The present invention addresses the problems associated with hydrogen peroxide compositions for treating fungal infections such as athletes foot and nail fungus but unexpectedly actually promotes healing of wounds while also disinfecting.

It is, therefore, a principal object of the subject invention to provide an easily applied gel that can be utilized to treat fungal infections such as athletes foot and nail fungus.

It is a further object of the invention is to disinfect wounds of the skin and promote faster healing.

It is a further object of the present disclosure to provide a process for producing a stable disinfecting gelled composition that promotes healing containing hydrogen peroxide and a smectite clay comprised of mixing said smectite gelling agent, which is free of any heavy metal contamination, under conditions where all mixing equipment is free of heavy metals.

It is a further object of the present disclosure to provide a process for producing a compound where the mixing is accomplished by tumbling or rolling closed containers, with cowls type dissolver, paddle mixer, colloid mill, or ultrasonication.

It is a further object of the present disclosure to provide a process for producing a compound where all mixing equipment and containers is coated with a coating that is free of heavy metals.

It is a further object of the present disclosure to provide a stable disinfecting gelled composition that promotes healing that is comprised of hydrogen peroxide solution in water and a smectite gelling agent.

It is a further object of the present disclosure to provide a composition where the hydrogen peroxide concentration is between 1 and 30%.

It is a further object of the present disclosure to provide a composition where the hydrogen peroxide concentration is between 1 and 6%.

It is a further object of the present disclosure to provide a composition where the hydrogen peroxide concentration is more preferably between 2 and 5%.

It is a further object of the present disclosure to provide a composition where the smectite gelling agent is a synthetic aluminosilicate with the general formula (Na)×(Al2-xMgx)2Si4O10(OH)2.n(H2O) where x is between 0.1 and 0.5 that is free of any heavy metal contaminants.

It is a further object of the present disclosure to provide a composition where the smectite gelling agent is a synthetic magnesiosilicate of the formula Nax(Mg3-x Lix)3Si4O10(OH)2 where x is between 0.1 and 0.5 that is free of any heavy metal contaminants.

It is a further object of the present disclosure to provide a composition where the OH is substitute wholly or partially with fluoride ion.

It is a further object of the present disclosure to provide a composition where the OH is substitute wholly or partially with fluoride ion.

It is a further object of the present disclosure to provide a composition where the concentration of smectite gelling agent is between 1 and 7%.

It is a further object of the present disclosure to provide a composition where the concentration is more preferably between 2 and 3%.

### BRIEF DESCRIPTION OF THE DRAWINGS

The novel features believed characteristic of the disclosed subject matter will be set forth in any claims that are filed later. The disclosed subject matter itself, however, as well as a preferred mode of use, further objectives, and advantages thereof, will best be understood by reference to the following detailed description of an illustrative embodiment when read in conjunction with the accompanying drawings, wherein:

FIG. 1 depicts Nail fungus infection prior to treatment.

FIG. 2 depicts Nail after treatment for 5 weeks showing marked improvement and healthy nail bed.

### DETAILED DESCRIPTION OF ILLUSTRATIVE EMBODIMENTS

The invention is a disinfecting gel that also unexpectedly promotes healing. The gel is an excellent disinfectant and can be used to treat fungal infections. The gel is applied topically and in the case of nail fungus eliminates the fungus without internal medications that have potential serious side effects on the liver. The most unexpected facet of the invention is the promotion of healing when applied to a wound. The medical literature recommends against applying hydrogen peroxide to wounds as a disinfectant because it is cytotoxic to healthy cells as well as bacteria which slows healing. The gelled hydrogen peroxide of this invention not only disinfects the wound but speeds up healing by 3 to 4 days by skipping the inflammatory stage of healing.

The phases of wound healing are:
Hemostasis
Inflammation
Proliferation or Granulation
Remodeling or Maturation

### Hemostasis

Once the source of damage to a house has been removed and before work can start, utility workers must come in and cap damaged gas or water lines. So too in wound healing damaged blood vessels must be sealed. In wound healing the platelet is the cell which acts as the utility worker sealing off the damaged blood vessels. The blood vessels themselves constrict in response to injury but this spasm ultimately relaxes. The platelets secrete vasoconstrictive substances to aid in this process but their prime role is to form a stable clot sealing the damaged vessel. Under the influence of ADP (adenosine diphosphate) leaking from damaged tissues the platelets aggregate and adhere to the exposed collagen. They also secrete factors which interact with and stimulate the intrinsic clotting cascade through the production of thrombin, which in turn initiates the formation of fibrin from fibrinogen. The fibrin mesh strengthens the platelet aggregate into a stable hemostatic plug. Finally platelets also secrete cytokines such as platelet-derived growth factor (PDGF), which is recognized as one of the first factors secreted in initiating subsequent steps.

Hemostasis occurs within minutes of the initial injury unless there are underlying clotting disorders.

### Inflammation Phase

Clinically inflammation, the second stage of wound healing presents as erythema, swelling and warmth often associated with pain, the classic “rubor et tumor cum calore et dolore”. This stage usually lasts up to 4 days post injury. In the wound healing analogy the first job to be done once the utilities are capped is to clean up the debris. This is a job for non-skilled laborers. These non-skilled laborers in a wound are the neutrophils or PMN's (polymorphonucleocytes). The inflammatory response causes the blood vessels to become leaky releasing plasma and PMN's into the surrounding tissue. The neutrophils phagocytize debris and microorganisms and provide the first line of defense against infection. They are aided by local mast cells. As fibrin is broken down as part of this clean-up the degradation products attract the next cell involved. The task of rebuilding a house is complex and requires someone to direct this activity or a contractor. The cell which acts as “contractor” in wound healing is the macrophage. Macrophages are able to phagocytize bacteria and provide a second line of defense. They also secrete a variety of chemotactic and growth factors such as fibroblast growth factor (FGF), epidermal growth factor (EGF), transforming growth factor beta (TGF- and interleukin-1 (IL-1) which appears to direct the next stage.

### Proliferative Phase (Proliferation, Granulation and Contraction)

The granulation stage starts approximately four days after wounding and usually lasts until day 21 in acute wounds depending on the size of the wound. It is characterized clinically by the presence of pebbled red tissue in the wound base and involves replacement of dermal tissues and sometimes subdermal tissues in deeper wounds as well as contraction of the wound. In the wound healing analogy once the site has been cleared of debris, under the direction of the contractor, the framers move in to build the framework of the new house. Sub-contractors can now install new plumbing and wiring on the framework and siders and roofers can finish the exterior of the house. The “framer” cells are the fibroblasts which secrete the collagen framework on which further dermal regeneration occurs. Specialized fibroblasts are responsible for wound contraction. The “plumber” cells are the pericytes which regenerate the outer layers of capillaries and the endothelial cells which produce the lining. This process is called angiogenesis. The “roofer” and “sider” cells are the keratinocytes which are responsible for epithelialization. In the final stage of epithelializtion, contracture occurs as the keratinocytes differentiate to form the protective outer layer or stratum corneum.

### Remodeling or Maturation Phase

Once the basic structure of the house is completed interior finishing may begin. So too in wound repair the healing process involves remodeling the dermal tissues to produce greater tensile strength. The principle cell involved in this process is the fibroblast. Remodeling can take up to 2 years after wounding and explains why apparently healed wounds can break down so dramatically and quickly if attention is not paid to the initial causative factors.

It is believed that the synthetic smectite clay is responsible for mediating the deleterious effects of hydrogen peroxides on healthy cells but also must sequester some of the signaling molecules that initiate the inflammation phase of healing. It further appears that as the water in the gel evaporates the smectite clay forms a protective layer over the wound. This coating of clay also keeps the wound humidified due to the natural tendencies of smectites to absorb moisture from the air. In addition the wound heals with less scarring so the clay also seems to play a role in promoting third and fourth stages of healing. This could be due to the fact that when the wound skips the inflammation stage less trauma is experienced by the wound.

The following are examples of the efficacy of the invention.

### EXAMPLE 1

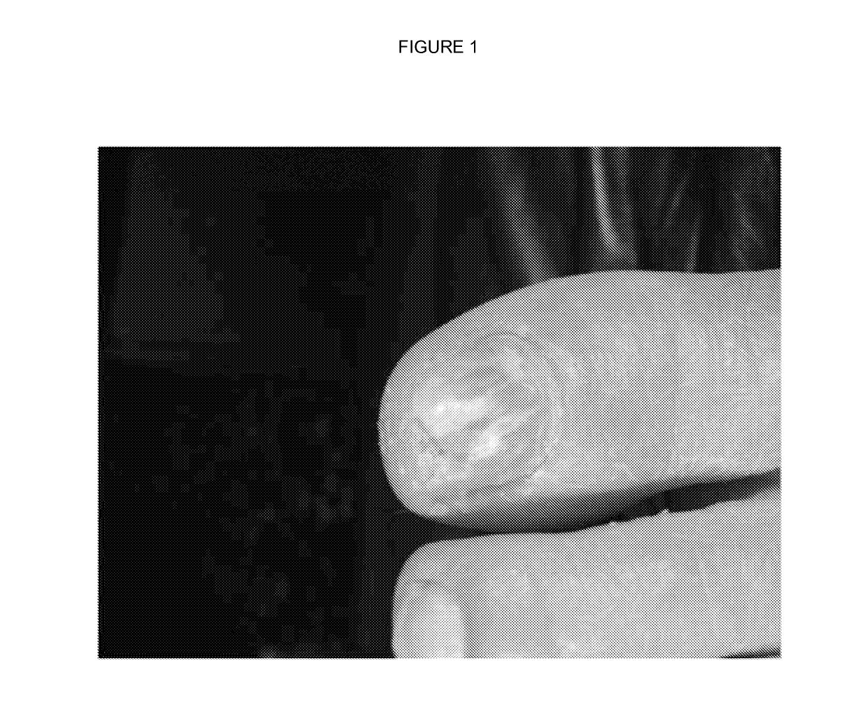

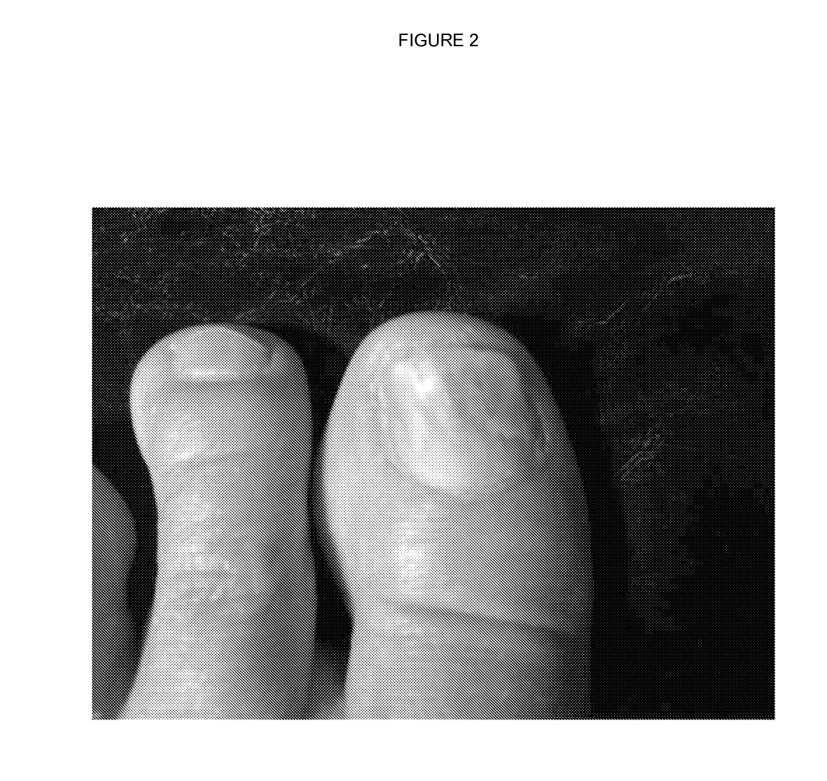

In this example the person had experienced chronic toenail fungus. The person had tried every treatment option available without eliminating the fungus. The person started applying the gel, containing 3% hydrogen peroxide and 2.5% smectite gelling agent, daily to the nail. FIGS. 1 and 2 contains a comparison of the nail before treatment and after five weeks. It can clearly be seen that health cuticle has been restored and the diseased nail has moved away from the cuticle.

### EXAMPLE 2

A second person had experienced athletes foot periodically over the years. The outbreaks resulted in an itchy rash, bleeding cracks between the toes, and blisters on the soles of the foot. In past attempts to treat the malady with over the counter drugs would result in cessation of symptoms in about three weeks. The person applied the gel containing 3% hydrogen peroxide and 2.5% smectite gelling agent of this invention twice a day. The symptoms disappeared in only three days.

### EXAMPLE 3

A person who was clearing brush encountered briers and received multiple scratches on his arms. The gel of this invention, containing 3% hydrogen peroxide and 2.5% smectite gelling agent, was applied to only one arm while the other was left untreated as a control. The cuts on the control arm became red and inflamed and did not go into the second stage of healing for about three days. The control arm cuts required about two weeks to heal. The cuts treated with the gel of this invention never became inflamed and began healing almost immediately. They healed in a week with almost no scarring.

Similar healing of wounds have been experienced by dozens of individuals when employing the gel of this invention.

### EXAMPLE 4

An individual was working on his barn and got a splinter in his finger. He though that he had extracted all of the splinter but the next day the wound was inflamed and a small pustule had formed. A small amount of the gel of this invention, containing 3% hydrogen peroxide and 2.5% smectite gelling agent, was applied to the wound and within an hour the inflammation began to subside. The next day the inflammation was completely gone and the wound completely healed in four days.

These examples are meant to be illustrative and are not exhaustive.

What is claimed is:

1. A process for producing a stable disinfecting gelled composition that promotes healing containing hydrogen peroxide and a smectite clay comprised of mixing said smectite gelling agent, which is free of any heavy metal contamination, under conditions where all mixing equipment is free of heavy metals.

2. A process as claimed in claim 1 where the mixing is accomplished by tumbling or rolling closed containers, with cowls type dissolver, paddle mixer, colloid mill, or ultrasonication.

3. A process as in claim 1 where all mixing equipment and containers is coated with a coating that is free of heavy metals.

4. A stable disinfecting gelled composition that promotes healing that is comprised of hydrogen peroxide solution in water and a smectite gelling agent.

5. A composition as claimed in claim 4 where the hydrogen peroxide concentration is between 1 and 30%.

6. A composition as claimed in claim 4 where the hydrogen peroxide concentration is between 1 and 6%.

7. A composition as claimed in claim 4 where the hydrogen peroxide concentration is more preferably between 2 and 5%.

8. A composition as claimed in claim 4 where the smectite gelling agent is a synthetic aluminosilicate with the general formula (Na)x(Al2-xMgx)2Si4O10(OH)2.n(H2O) where x is between 0.1 and 0.5 that is free of any heavy metal contaminants.

9. A composition as claimed in claim 4 where the smectite gelling agent is a synthetic magnesiosilicate of the formula Nax(Mg3-x Lix)3Si4O10(OH)2 where x is between 0.1 and 0.5 that is free of any heavy metal contaminants.

10. A composition as claimed in claim 8 where the OH is substitute wholly or partially with fluoride ion.

11. A composition as claimed in claim 9 where the OH is substitute wholly or partially with fluoride ion.

12. A composition as claimed in claim 4 where the concentration of smectite gelling agent is between 1 and 7%.

13. A composition as claimed in claim 4 where the concentration is more preferably between 2 and 3%.
